(19) United States
(12) Patent Application Publication
(10) Pub. No.: US 2011/0036013 A1
(43) Pub. Date: Feb. 17, 2011

(54) REMOVAL OF LIQUID ASH AND ALKALIS FROM SYNTHESIS GAS

(75) Inventors: Domenico Pavone, Bochum (DE); Michael Rieger, Dortmund (DE); Ralf Abraham, Bergkamen (DE)

Correspondence Address: MARSHALL & MELHORN, LLC, FOUR SEAGATE - EIGHTH FLOOR, TOLEDO, OH, 43604, US

(73) Assignee: UHDE GMBH, Dortmund (DE)

(21) Appl. No.: 12/735,205

(22) PCT Filed: Dec. 22, 2008
(86) PCT No.: PCT/EP2008/010995

§ 371 Date: Oct. 13, 2010

(30) Foreign Application Priority Data
Dec. 22, 2007 (DE) 10 2007 063 118.0
Mar. 7, 2008 (DE) 10 2008 013 179.2

(51) Int. Cl. C10J 3/72; C07C 1/02; B01J 19/00; F02C 3/20; H02K 7/18

(52) U.S. Cl. 48/210; 518/703; 75/392; 252/373; 48/197 R; 422/198; 60/780; 290/52

(57) ABSTRACT

A process for the production of synthesis gas by gasification with the aid of air or oxygen or oxygen-saturated air as well as water vapor, a solid or liquid carbon-bearing fuel material being fed to a reactor, is shown. The fuel material is converted with the aid of air or oxygen or oxygen-saturated air and water vapor at an elevated temperature into a synthesis gas essentially consisting of hydrogen, carbon dioxide and carbon monoxide; the reaction also yields mineral slag droplets which are removed from the reactor separately from the synthesis gas obtained, and the synthesis gas being discharged from the reactor in any random direction desired; the vaporous alkalis contained in the synthesis gas come into contact with a getter ceramic material so that they separate from the synthesis gas and hence, the synthesis gas can be sent to a slag separation device without previous cooling step and the slag droplets being withdrawn from the said device as liquid slag.

The present invention relates to a process for the production of synthesis gas from a carbon-bearing fuel material, such as any type of coal, coke, petroleum coke, biomass, but also emulsions, Orimulsion, etc. The process in accordance with the present invention permits easy purification of the synthesis gas directly after the production of the said gas without cooling-down step. This facilitates the exploitation of the thermal energy of the gas. This invention also encompasses a device required to utilize this process as well as the application techniques of the getter ceramics used.

When synthesis gas is produced from a carbon-bearing fuel material, the said fuel material is converted to a gas bearing water vapour or water vapour and oxygen, in an appropriate reactor. Apart from the synthesis gas produced, this process also yields liquid mineral ash and slag which, as a rule, consist of aerosols and droplets. Some types of the liquid ash materials partly evaporate and form alkali vapours. These components are indeed detrimental to a further utilization because processing them in the downstream process equipment may entail damage to the equipment or impair the process.

In many cases synthesis gas is used to produce important chemicals, such as ammonia or methanol. The components that are detrimental to or even impair the processing must be removed from the synthesis gas prior to carrying out the necessary process steps. For this purpose the synthesis gas is often mixed with a cooler foreign medium to exploit the high thermal energy contained in the gas. The foreign medium used in this case is, as a rule, water. But it is also possible to utilize other media, such as nitrogen or carbon dioxide. In this process step, the synthesis goes undergoes a considerable cooling (quenching) and is often followed by further process steps which frequently require that the synthesis gas be cooled even further. Such steps are, for example, scrubbing processes to remove sour gas.

During these process steps, a major part of the useful thermal energy contained in the synthesis gas gets lost. For further exploitation, however, high temperatures are often needed. The respective synthesis gas must then be re-heated which requires much energy. Therefore, it would be more convenient to enable further processing of the synthesis gas thus obtained without being subjected to cooling. As the gas is under a high pressure directly after production, too, it is also possible to deploy a turbine for the recovery of kinetic rotation energy. This energy supplied by the turbine could for example be exploited to generate electric power or to drive plant machinery. This method permits an efficient process for synthesis gas production. Hence, such a process allows a combined production of synthesis gas and electric power.

A prerequisite for such a deployment, however, is that the synthesis gas obtained can be fed to the turbine without a cooling step. It would be beneficial for such a process if the liquid and gaseous pollutants of the synthesis gas were removed from the gas without cooling and without changing its physical state, because liquid droplets and corrosive vapours might cause erosion and corrosion which entail damage to the turbine blades.

Document U.S. Pat. No. 4,482,358 A describes a process for the production of synthesis gas, in which the synthesis gas passes through in a cyclone-type vessel packed with a circulating solids bed of various grain sizes. When flowing through the solids bed, the entrained solid and slag particles solidify and are removed from the system. The deployment of a slag crusher permits a re-use of the solids thus reduced to adequate grain size. The gas as well as the reduced slag particles can be sent to heat exchangers utilized for driving the power generation turbine. Prior to sending it to the pressure vessel, the synthesis gas undergoes a cooling process with the aid of water. A disadvantage of the system is that water must be used for cooling the synthesis gas. A further demerit of this process is the necessity that the steam required to drive the machine must be generated. The said document does not describe a separation of the metallic compounds from the synthesis gas.

Document EP 412 591 B1 describes a process for the separation of alkali and heavy metal compounds from hot gases. The latter are obtained as combustion gases while burning fossil fuel materials and the combustion gases are used for driving a power generation gas turbine. In order to preclude a corrosion of the gas turbine due to the metallic salts contained in the combustion gases, the latter are treated with a sorption agent prior to being fed to the gas turbine, the said agent becoming suspended in the stream of hot gases. The state of the suspension is described as a type of flue dust cloud or an expanded fluidized bed of the sorption agent. The sorption agent may consist of silicium dioxide, aluminium oxide, magnesium aluminosilicate or calcium aluminosilicate. A combination of the alkalis separation with the production of synthesis gas is not described, nor does the said document describe the removal of flue ash or liquefied slag from the hot gases.

The objective of the present invention, therefore, is to provide a process and a device which permit the removal of liquid slags and alkalis entrained by the synthesis gas originating from a gasification process, yet without the necessity to cool down or expand the gases. The deployment of a turbine for the generation of rotation energy must be such that there is no formation of incrustations nor a corrosive or erosive attack on the material due to the hot synthesis gas.

The said objective of the invention is achieved by a process for the production of synthesis gas by way of gasification with the aid of air or oxygen or oxygen saturated air and hydrogen, in accordance with the technical criteria listed below:
A solid or liquid carbon-bearing fuel material is fed to a reactor in which a conversion of the fuel material to synthesis gas takes place with the aid of air or oxygen or oxygen-bearing air as well as hydrogen at an elevated temperature, the said synthesis gas mainly consisting of hydrogen, carbon dioxide and carbon monoxide, and
the reaction yields mineral slag droplets which are removed from the reactor separately from the synthesis gas obtained, and
the synthesis gas thus produced being discharged from the reactor in a random direction,
the vaporous alkalis contained in the synthesis gas being separated from the synthesis gas by coming into contact with a getter ceramic packing, and
without previous cooling, the synthesis gas is sent to a slag separation device from which the slag droplets are withdrawn in the form of liquid slag.

Prior to being processed, the said fuel materials are preferably treated in a device suited for reducing the grain size of the material particles. In this case it is possible to use, for example, a ball mill or a vertical mill, but a shredder or milling machine may also be suitable. This operation is needed to obtain the grain size diameter required for the gasification process. The burning gas utilized is especially water vapour bearing air which mainly reacts with the carbon content of the fuel material and thus forms carbon monoxide and hydrogen. A feature is that the burning gas is fed at an elevated pressure. The fuel material is preferably fed pneumatically to the gasification reactor. But it is also possible to feed the fuel material to the said reactor by means of a screw conveyor or a belt conveyor. Whenever the fuel material is available in the form of slurry or emulsion it can also be pumped to the reactor.

The synthesis gas is discharged at a different point of the reactor, but preferably at a lateral point. However, it is also possible to discharge it at any point of the reactor. The discharge of the liquid components must be carried out directly afterwards. In accordance with embodiments of the invention, the slag separation device is a cyclone-type device in which the hot gas performs a circular motion such that the major part of the slag contained in the gas precipitates on the walls due to the centrifugal force. Additionally or as an option, the slag separation device can be provided with a bed of bulk material in which the slag separates from the gas. The said bulky packing can be integrated into the cyclone; document DE 43 36 100 C1 describes such a type of design.

Further embodiments of the invention relate to the separation of the vaporous alkalis. For this purpose it is possible to add the getter ceramic material as powder to the fuel material, the getter ceramic stuff in the gasification chamber coming into contact with the synthesis gas produced and the removal of the alkalis from the gas thus taking place in the gasification chamber. Additionally or as an option, the getter ceramic material may be provided as bulk material in a device arranged downstream of the slag separation unit to put the synthesis gas into contact with it, the removal of the alkalis from the gas being effected in this downstream device. Furthermore, the getter ceramic material can even be admixed downstream of the gasification step. The addition of the getter ceramic material can be effected by injection or by similar methods.

Further embodiments of the invention relate to the process parameters of the gasification. Any material that contains solid carbon-bearing substances and is suitable for gasification and conversion with the aid of a water vapour or oxygen bearing gas can be used as fuel material. This particularly applies to any type of fine-grain coal with a typical grain size diameter. Hence, any coal type is applicable, for example, crushed hard coal or lignite. Any fine-grain plastic material, petroleum coke, biological fuel material, such as chopped wood or bitumen or other biomass are suitable. The fuel material may also be fed in liquid form as, for example, slurry or emulsions of fine-grain substances, which also include Orimulsion or, as a rule, viscous fuel materials, too. Finally it can be stated that any substances are suitable which can be converted to synthesis gas at elevated temperatures, hence essentially consisting of carbon monoxide and hydrogen. The gasification temperature must be selected from a range of 800 to 1800° C., the pressure from a range of 0.1 to 10 MPa.

Further embodiments of the invention relate to further treatment options of the produced synthesis gas. Thus it is possible to provide downstream of the slag and alkali separation from the synthesis gas originating from the gasification unit, a gas scrubber for removing sour gases, for example, for the separation of sulphur-bearing components with the aid of a chemisorbent.

Further embodiments of the invention relate to further application options of the produced synthesis gas. It is in fact possible to provide downstream of the slag and alkali separation from the synthesis gas originating from the gasification unit, piping for sending the synthesis gas through a hot gas turbine, the latter being coupled to a generator for electric power generation or to a compressor for the compressed burning air required for the gasification. As the hot gas supplies power output, the hot gas cools down. After further energy recovery, for example steam generation, the synthesis gas thus obtained can be exploited for the synthesis of chemical products, the production of metals by the direct reduction method or for power generation in a gas turbine.

The present invention also encompasses a device for the production of synthesis gas by gasification in accordance with the process described above, which includes a reactor suitable for the gasification of carbon-bearing fuel materials at high temperatures and equipped with a device for the feed of air or oxygen or oxygen-bearing air and of hydrogen, the said reactor also having a reaction chamber for the conversion of carbon-bearing fuel materials and at least, a single stage hot-gas cyclone being arranged directly downstream of the reactor, the said cyclone being provided with a slag removal device for liquid slag or a device with a bulky bed installed at this point and with a removal device for liquid slag or both devices, the order of installation being freely selectable.

In accordance with further embodiments of the invention, it is possible to install directly downstream of the slag removal device, a further device packed with bulky getter ceramic materials and a hot gas turbine being integrated behind this packed device.

The invention also includes the use of getter ceramic materials. With regard to the materials to be used for this purpose, it is envisaged that the getter ceramic material consists of either silicium dioxide or silicate or aluminate or aluminium oxide or compounds or mixtures thereof or any compounds of oxide or non-oxide ceramic material. Moreover, they can contain transitional metal compounds. According to a preferred embodiment of the invention, the getter ceramic material is formed from aluminosilicates, specific preference being given to kaoline, emathlite, bentonite and montmorillonite.

Further types of embodiments relate to the form or state of getter ceramic material: If the getter ceramic material is added to the fuel material, it is powder-type, in any other case of application it is of highly porous solid particles, i.e. a layer of bulky material packed in the alkalis separator. In the cases of highly porous solid particles, the following forms are suitable: balls, saddle packings, Raschig rings, pall rings or cylindrical types, or even any other shape selected. The grain size diameter, as a rule, ranges from 2 mm to 100 mm, preferably 20 to 40 mm, but especially preferred 30 mm.

The device as described in the present invention is illustrated on the basis of the attached drawing, the type of configuration not being restricted to the example depicted in the drawing.

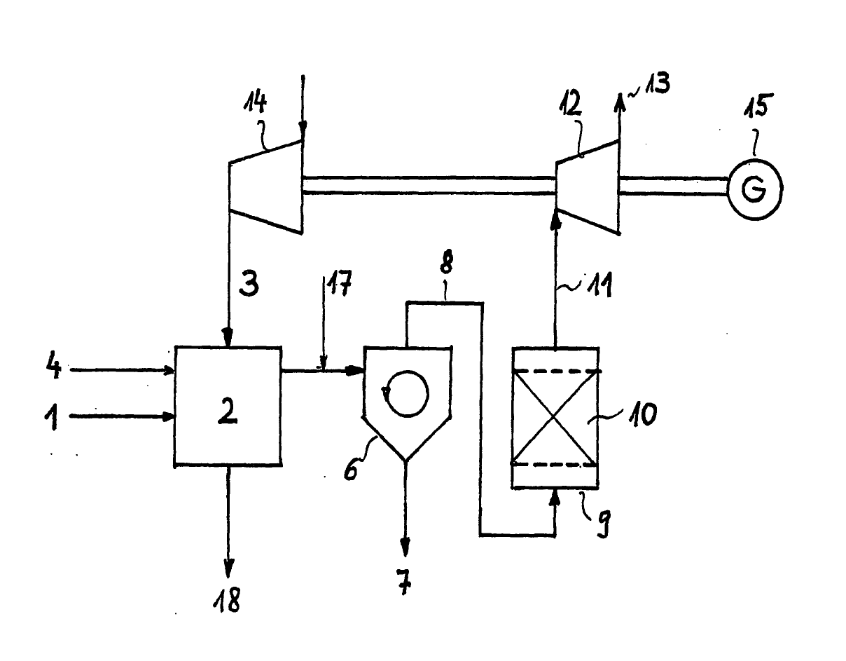

FIG. 1 shows a simplified process flow diagram of the process in accordance with the invention for the production and treatment of synthesis gas, the inherent energy of which is used for the generation of electric power. The fuel material 1 is fed to the gasification reactor 2 and converted therein to a synthesis gas 5 laden with slag droplets and alkalis, with the aid of compressed oxygen saturated air 3. The gasifier can be equipped with a slag outlet. The additives can be fed downstream of the gasifier. The synthesis gas 5 is sent to a cyclone 6 in which it is freed from the slag droplets and, if any, from the alkalis. The slag 7 is withdrawn in liquid form. The synthesis gas 8 thus freed from slag is piped to the vessel 9 packed with bulky getter ceramic material 10, the gas thus being freed from alkalis. The hot gas 11 thus purified is then fed to a hot gas turbine 12 in which it is expanded. The synthesis gas 13 expanded and thus cooled down is branched off for further applications. The drive shaft power output of the hot gas turbine 12 is utilized for driving the compressor 14 and the generator 15. The compressor 14 compresses the oxygen saturated air 16, the latter being sent to the gasification reactor 2.

The following set of figures serves to illustrate the efficiency of the system in accordance with the invention. When coal is gasified, an amount of 8 to 40 g/m³ (on the basis on STP) of liquid slag particles and a quantity of alkali vapours of up to 200 mg/m³ (on the basis of STP) are released in the raw gas. When entering into the cyclone 6, the respective portions still contained in the synthesis gas 5 are as follows: about 4 to 20 g/m³ (on the basis of STP) of liquid slag particles and up to 90 mg/m³ (on the basis of STP) of alkali vapours. At the entry of the hot gas turbine 12, the hot gas 11 merely contains an amount of liquid slag particles of 5 mg/m³ (on the basis of STP) and a quantity of alkali vapours of less than 0.013 mg/m³ (on the basis of STP).

### KEY TO REFERENCED ITEMS

1 Fuel material
2 Gasification reactor
3 Compressed, oxygen-saturated air
4 Water vapour
5 Synthesis gas
6 Cyclone
7 Slag removal
8 Synthesis gas freed from slag
9 Vessel
10 Bulky getter ceramic material
11 Hot gas
12 Hot gas turbine
13 Cooled synthesis gas
14 Compressor
15 Generator
16 Oxygen-saturated air
17 Addition of additives
18 Slag outlet

As an alternative, it is also possible to understand the referenced item 1 to be a fuel material with additive for alkalis removal and the referenced item 6 to be a bulky bed or a cyclone with a respective bulky bed.

1-27. (canceled)

28. A process for the production of synthesis gas by gasification with the aid of air or oxygen or oxygen-saturated air as well as hydrogen, comprising:
a solid or liquid carbon-bearing fuel material is fed to a reactor for the production of synthesis gas by way of gasification with the aid of air or oxygen or oxygen-saturated air as well as hydrogen at an elevated temperature, the synthesis gas mainly comprising hydrogen, carbon dioxide and carbon monoxide;
the reaction yields mineral slag droplets which are removed from the reactor separately from the synthesis gas obtained; and
the synthesis gas thus produced being discharged from the reactor in a random direction; wherein
the vaporous alkalis contained in the synthesis gas are separated from the synthesis gas by coming into contact with a getter ceramic packing, and
without previous cooling, the synthesis gas is sent to a slag separation device from which the slag droplets are withdrawn in the form of liquid slag.

29. The process according to claim 28, wherein the slag separation device is a cyclone-type device in which the hot gas performs a circular motion such that the major part of the slag contained in the gas precipitates on the walls due to the centrifugal force.

30. The process according to claim 28, wherein the slag separation device is provided with a bed of bulk material in which the slag separates from the gas.

31. The process according to claim 28, wherein the getter ceramic material is added as additive to the fuel material, the getter ceramic stuff in the gasification chamber coming into contact with the synthesis gas produced and the removal of the alkalis from the gas thus taking place in the gasification chamber.

32. The process according to claim 28, wherein the getter ceramic material may be provided as bulk material in a separation device arranged downstream of the slag separation unit to put the synthesis gas into contact with it, the removal of the alkalis from the gas being effected in this downstream device.

33. The process according to claim 28, wherein coal, coal emulsion, coal slurry, petroleum coke, biological fuel materials or plastic materials in fine-grain form are suitable as fuel material.

34. The process according to claim 28, wherein the gasification takes place at a temperature of 800 to 1800° C.

35. The process according to claim 28, wherein the gasification take place at a pressure of 0.1 to 10 MPa.

36. The process according to claim 28, wherein a chemisorbent for the removal of sulphur-bearing components is added to the synthesis gas originating from gasification and already freed from slag and alkalis.

37. The process according to claim 28, wherein upon separation of the slag, alkalis and, if any, sulphur-bearing substances, the hot synthesis gas is sent to a hat gas turbine.

38. The process according to claim 37, wherein a power generator is coupled to the hot gas turbine in order to produce electric energy.

39. The process according to claim 37, wherein a compressor is coupled to the hot gas turbine in order to compress the air required for the gasification.

40. The process according to claim 28, wherein the synthesis gas thus obtained is exploited for the synthesis of chemical products, the production of metals by the direct reduction method or for power generation.

41. A device for the production of synthesis gas by way of gasification in accordance with the process of claim 29, comprising:
a reactor suitable for the gasification of carbon-bearing fuel materials at high temperatures;
the reactor comprising:
a device for the feed of air or oxygen or oxygen-bearing air and of hydrogen; and
a reaction chamber for the conversion of carbon bearing fuel materials with the aid of a water vapour or water vapour and oxygen-bearing gas; and
at least a single-stage hot gas cyclone arranged directly downstream of the reactor and provided with a removal device for liquid slag.

42. A device for the production of synthesis gas by way of gasification in accordance with the process of claim 29, comprising:
a reactor suitable for the gasification of carbon-bearing fuel materials at high temperatures;
the reactor comprising:
a device for the feed of air or oxygen or oxygen-bearing air and of hydrogen; and
a reaction chamber for the conversion of carbon bearing fuel materials with the aid of a water vapour or water vapour and oxygen-bearing gas; and
at least a single-stage hot gas cyclone is arranged directly downstream of the reactor and provided with a bulky bed and a removal device for liquid slag.

43. A device for the production of synthesis gas by way of gasification in accordance with the process of claim 30, comprising:
a reactor suitable for the gasification of carbon-bearing fuel materials at high temperatures;
the reactor comprising:
a device for the feed of air or oxygen or oxygen-bearing air and of hydrogen, and
a reaction chamber for the conversion of carbon bearing fuel materials with the aid of a water vapour or water vapour and oxygen-bearing gas; and
a device arranged directly downstream of the reactor is provided with a bulky bed and a removal device for liquid slag.

44. The device according to claim 41, wherein at least one single-stage hot gas cyclone and a device provided with a bulky bed are arranged directly downstream of the reactor, each of the two downstream devices having a removal device for liquid slag.

45. A device for the production of synthesis gas by way of gasification in accordance with the process of claim 33, comprising:
a reactor suitable for the gasification of carbon-bearing fuel materials at high temperatures;
the reactor comprising:
a device for the feed of air or oxygen or oxygen-bearing air and of hydrogen; and
a reaction chamber for the conversion of carbon bearing fuel materials with the aid of a water vapour or water vapour and oxygen-bearing gas;
at least a single-stage hot gas cyclone arranged directly downstream of the reactor and provided with a removal device for liquid slag; and
directly downstream of the slag removal device, an additional device packed with a bulky getter ceramic material.

46. The device according to claim 41, further comprising a hot gas turbine installed downstream of the device for the purification of the synthesis gas stream to eliminate slag and alkalis.

47. The process according to claim 28, wherein the getter ceramic material primarily comprises: silicium dioxide or silicate or aluminate or aluminium oxide or compounds or mixtures thereof, or any compounds of oxide and non-oxide ceramic material.

48. The process according to claim 47, wherein the getter ceramic material contains transitional metal compounds.

49. The process according to claim 47, wherein the getter ceramic material is formed from aluminosilicates, specific preference being given to kaoline, emathlite, bentonite and montmorillonite.

50. The process according to claim 47, wherein the getter ceramic material comprises highly porous solid particles packed in the form of a bulky layer in the alkalis separator.

51. The process according to claim 50, wherein the highly porous solid particles are packed in the following forms: balls, saddle packings, Raschig rings, pall rings or cylindrical types.

52. The process according to claim 49, wherein the getter ceramic material is packed or hanged in the alkalis separator, i.e. in the form of highly porous ceramic material pre-formed.

53. The process according to claim 50, wherein the getter ceramic material has a grain size diameter of 2 to 100 mm.

54. The process according to claim 50, wherein the getter ceramic material has a grain size diameter of 20 to 40 mm.
